(19) United States
(12) Patent Application Publication
(10) Pub. No.: US 2006/0004393 A1
(43) Pub. Date: Jan. 5, 2006

(54) Percutaneous anastomosis connection system

(76) Inventor: Paul D. Amarant, Davie, FL (US)

Correspondence Address: BRINKS HOFER GILSON & LIONE/INDY/COOK, ONE INDIANA SQUARE, SUITE 1600, INDIANAPOLIS, IN, 46204-2033, US

(21) Appl. No.: 11/121,606

(22) Filed: May 4, 2005

Related U.S. Application Data
(60) Provisional application No. 60/570,673, filed on May 13, 2004.

(51) Int. Cl. A61B 17/08

(52) U.S. Cl. 606/153

(57) ABSTRACT

A system for providing an end-to-end anastomosis connection between two vessels. The system comprises first and second hollow tubes. Each of the tubes has a first end that is attachable to a free end of a separate one of the two vessels, and a second end that comprises a connector. The respective connectors are sized and shaped for engagement to each other, such that a fluid-tight connection can be established therebetween.

### RELATED APPLICATION

The present patent document claims the benefit of the filing date under 35 U.S.C. §119(e) of Provisional U.S. patent application Ser. No. 60/570,673, filed May 13, 2004, which is hereby incorporated by reference.

### BACKGROUND

1. Technical Field

The present invention relates to connectors for use in connecting tubular conduits, and more particularly, to a percutaneous anastomosis connection system for connecting medical grafts, body vessels, and the like.

2. Background Information

Anastomosis is the joinder of hollow vessels to create an internal communication between them. An anastomosis is generally created by a surgical procedure that joins two body vessels, vascular grafts, or a body vessel and a graft, in order to create or restore a pathway for fluid flow through the joined structure. Commonly, an anastomosis is created by vascular surgery to join two blood vessels, grafts, or a blood vessel and a graft, to create or restore blood flow therethrough.

Current devices and techniques exist which allow for open-ended surgical attachment of harvested blood vessels or grafts for purposes such as the avoidance of a vessel blockage, replacement of diseased vessels, and vascular access. Such techniques include sewing or otherwise attaching a vessel or graft between open ends of existing vessels. Examples of vessel pairs which are frequently joined by a vessel or graft include an internal mammary artery and a coronary vessel, the radial artery and cephalic vein, the brachial artery and cephalic vein, the brachial artery and basilica vein, the ulnar artery and a basilica vein, and a brachial artery and branches of the antecubital vein, among others.

It is generally preferred to join such vessels utilizing the patient's natural vessels. This connection may be between two natural vessels positioned in their natural place of orientation in the body, or alternatively, utilizing one or more natural vessels harvested from another portion of the patient's anatomy. Utilizing a vessel harvested from another portion of the patient's anatomy minimizes the possibility that the patient will experience incompatibility or rejection problems of the type that may occur when using graft materials that originate from an external source, or from using exogenous tissue. In addition, such harvested vessels provide a ready supply of biological tissue that has already proven to be biologically compatible with the patient.

At times, however, suitable body vessels may not be available for harvesting. In such cases, a synthetic vessel (e.g., TEFLON® or DACRON®) or an exogenous vessel may be used. Synthetic vessels have been found to be effective in many instances. However, such vessels have shown a greater propensity to become narrowed than do natural arteries or veins. Exogenous vessels may also be utilized in an appropriate case. However, there is a greater likelihood of patient incompatibility with such vessels when compared to vessels harvested from the patient.

Many different types of anastomosis connections between a vessel and a graft are known in the medical arts. For example, an anastomosis connection may be utilized to join vessels from the end of a graft to the side of a vessel, commonly referred to as an end-to-side connection. An anastomosis connection may also be utilized to join the end of a graft to the end of a vessel, commonly referred to as an end-to-end connection. A side-to-side connection of a vessel and a graft may also be established. This type of connection is commonly referred to as a fistula.

End-to-end connections are generally considered beneficial because they essentially mimic the normal flow of fluid through the natural vessel. With regard to such end-to-end connections, however, it is important to insure that a secure and leak-free connection be established. Prior art connection devices are at times unsecure, and also have been prone to leakage. While synthetic joinder materials are available, such materials are often complicated and difficult to use. A need exists for an improved device for end-to-end connection that provides a secure and leak-free connection, that is relatively easy for the surgeon to manipulate and insert, and that is cost-effective.

### BRIEF SUMMARY

The present invention addresses the problems of the prior art by providing a percutaneous anastomosis connection system for establishing a reliable connection between medical grafts and/or body vessels.

In one form thereof, the invention comprises a system for providing an end-to-end anastomosis connection between two vessels. The system comprises a first hollow tube having two ends, one of said ends being attachable to a free end of a first vessel, and the other end comprising a first connector. A second hollow tube has two ends, one of said ends being attachable to a free end of a second vessel, and the other end comprising a second connector. The first and second connectors are sized and shaped such that a mating connection can be established therebetween.

In another form thereof, the invention comprises a system for establishing an end-to-end anastomosis connection between two vessels, wherein the system comprises first and second ring members. The first ring member is connected to a free end of a first vessel, and the second ring member is connected to a free end of a second vessel. The first ring member includes a plurality of hook connectors, and the second ring member includes a plurality of loop connectors. The hook and loop connectors are sized and oriented such that a linkable connection can be established therebetween to establish a fluid communication between the first and second vessels.

In still another form thereof, the invention comprises a method for joining two vessels in an end-to-end anastomosis connection to establish a substantially leak-free fluid pathway therebetween. The method comprises the steps of: providing a connection system for the vessels, wherein the connection system comprises a first annular member and a second annular member, one end of the first annular member being attachable to a free end of a first vessel, and one end of the second annular member being to a free end of a second vessel, and wherein the other end of the first annular member includes a first connection member and the other end of the second connection member includes a second connection member, and wherein the first and second connection members are sized and shaped for establishing a mating connection therebetween; attaching the one end of the first annular member to the first vessel free end; attaching the one end of the second annular member to the second vessel free end; and establishing the mating connection between the first and second connection members.

### BRIEF DESCRIPTION OF THE DRAWINGS

FIG. 1 is a side view showing a percutaneous anastomosis system of the present invention in position to connect two body vessels;

FIG. 2 is a side view of an alternative embodiment of a percutaneous anastomosis system; and

FIG. 3 is a side view of another alternative embodiment of a percutaneous anastomosis system.

### DETAILED DESCRIPTION OF THE DRAWINGS AND THE PRESENTLY PREFERRED EMBODIMENTS

For the purposes of promoting an understanding of the principles of the invention, reference will now be made to the embodiments illustrated in the drawings, and specific language will be used to describe the same. It should nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

The present invention comprises a percutaneous anastomosis connection system for establishing an end-to-end anastomosis connection between two hollow structures in the body. The particular connections resulting from use of the inventive apparatus may be, for example, a graft-to-graft connection, a vessel-to-graft connection, or a vessel-to-vessel connection. For a vessel-to-vessel connection, the connection may be established between natural vessels, exogenous vessels, synthetic vessels, or any combination of the foregoing.

Although it is expected that the apparatus will normally be utilized to connect blood vessels, other body vessels may be joined to vessels, other body structures, grafts, synthetic or exogenous vessels. One non-limiting example of a connection of this type comprises the connection of the ureter vessel to the urethra. For applications in bodily systems, such as the circulatory system, ultrasound guidance can be utilized to help establish connection between the apparatus and other structures in the system, such as an artery, a vein, or both an artery and a vein. Those skilled in the art will appreciate that other bodily connections can be made using the apparatus and method of the present invention, and that medical guidance systems other than ultrasound may be utilized in an appropriate case, all of which are considered within the scope of the invention.

Such hollow body vessels, such as blood vessels, are joined in a manner to permit or restore fluid flow therebetween. The anastomosis connection provides a means to bridge the vessels within the body of a patient in end-to-end fashion. The term “vessel” is used herein in inclusive fashion to include body vessels or other hollow structures (both endogenous and exogenous), medical grafts, synthetics, and other segments that may be joined by the apparatus of the present invention.

Preferred embodiments of the invention will be further described in connection with the drawings. FIG. 1 shows a side view of a percutaneous anastomosis system of the present invention. This connection system illustrates two connector parts, namely male connector 10 and female connector 12. Male connector 10 is provided with external screw threads 14, and female connector 12 is provided with internal screw threads 16. Screw threads 14, 16 are sized and shaped to mate and establish a screw connection therebetween in well-known manner.

Connectors 10, 12 are generally formed of plastic or other biologically compatible material having sufficient strength to establish a reliable connection with another connector. Preferably, connectors 10, 12 are also formed from polymers or other materials having a relatively low durometer. In this way, sutures 22, 28 or like securement mechanisms can be easily passed through the wall of respective connectors 10, 12 in a manner to be described. Appropriate sutures 22, 28 acceptable for medical purposes are well known to those skilled in the art, and are commercially available.

In the non-limiting example shown in FIG. 1, an anastomosis connection is to be formed by the joinder of body vessel 20 and body vessel 26 in the manner shown. Vessels 20 and 26 can comprise, for example, blood vessels that are to be joined to establish, or re-establish, a fluid pathway through the joined vessels. Vessel 20 has free end 21, and vessel 26 has free end 27. Connector 10 is mounted to free end 21. In this embodiment, connector 10 is mounted to vessel free end 21 by sutures 22. Sutures 22 are threaded through both connector 10 and vessel 20 to establish a threaded connection therebetween in conventional fashion. Similarly, connector 12 is mounted to free end 27. In this case, sutures 28 are threaded through connector 12 and vessel 26 to establish the threaded connection. Those skilled in the art will appreciate that other conventional mounting mechanisms may be substituted for the sutures described herein.

The anastomosis connection between vessels 20, 26 may be established by rotating connectors 10, 12 in well-known manner to establish the screw connection therebetween. However, when a screw connection is established in this manner, the rotation of the connectors might cause the joined vessels to have a circumferential twist. Such a twist in one, or both, of the vessels may be undesirable, as it can result in the existence of an unintended twisting, or rotational, force between the vessels. Therefore, in order to minimize, or eliminate, this effect, one or both of the connectors may be pre-torqued before connection with the other connector. The pre-torque may be accomplished, for example, by twisting or winding one connector relative to the other prior to the screw connection. As the pre-torqued connector (and vessel end) unwinds from the pre-torqued condition, at least one of connectors 10, 12 rotates relative to the other such that threads 12, 14 screw together in normal fashion. In this way, the fluid connection may be established between vessels 20, 26 free from circumferential twist.

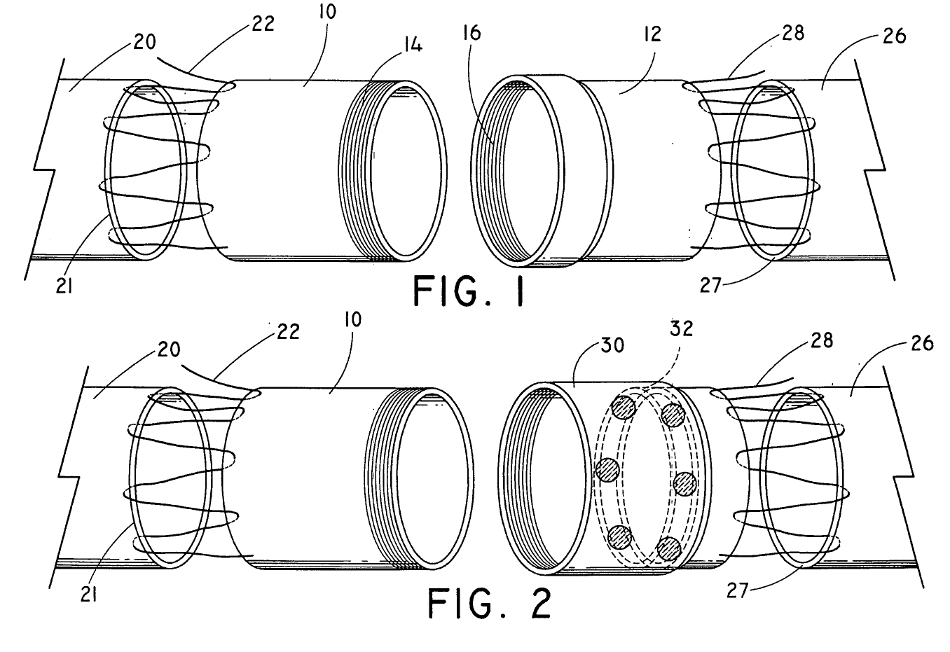

FIG. 2 illustrates an alternative embodiment of the inventive system. In this embodiment, vessels 20, 26 have respective free ends 21, 27 as before. Vessel 20 is connected to male connector 10 by sutures 22, as in the embodiment of FIG. 1. However, in this embodiment a rotatable structure, such as a ball bearing ring 32, is mounted at the free end of vessel 26. Ball bearing ring 32 is mounted by sutures 27 or other conventional attachment mechanism. Use of the ball bearing ring enables the threaded connector 30 to be rotated relative to connector 14 as the threaded connection is established, without requiring that the vessels be twisted or pre-torqued, and without resulting in a circumferential twist. Alternative structures to ball bearing ring may be substituted, as long as the desired rotational effect may be achieved.

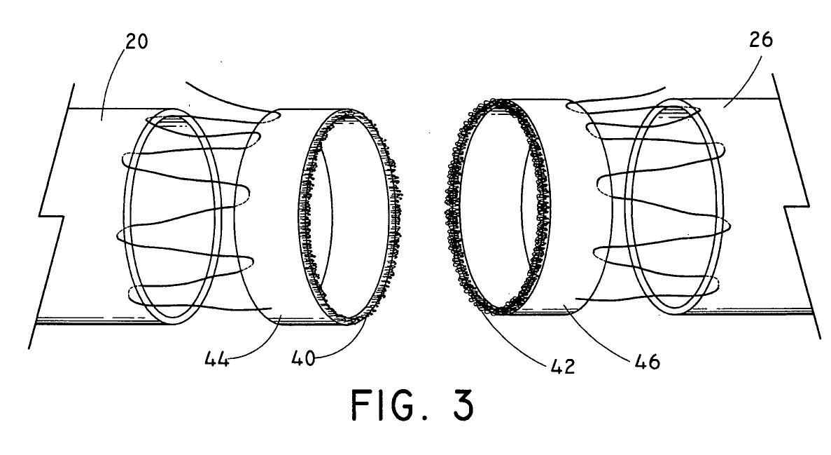

FIG. 3 illustrates another alternative embodiment of the inventive system. In this embodiment, a plastic tubular end with hook projections 40 is provided at the end of vessel 20, and a corresponding tubular end with loop projections 42 is provided at the end of vessel 26. Respective hook projections 40 and loop projections 42 are generally provided in nanoscale or microscale size on respective ring member substrates 44, 46. The projections and substrates are mounted to vessels 20, 26 by any convenient manner, such as by sutures as previously described. The hook/loop projections interlock with each other when pushed together, forming a tight, or even a water-tight, seal in well-known fashion. The circumference of substrates 44, 46, the lengths of projections 40, 42, and the spacing and distribution of the projections may be varied as desired to yield the desired attachment properties. In this embodiment, pre-torqueing or pre-twisting the vessels will normally not be necessary.

The use and insertion of the inventive percutaneous anastomosis connection system will now be described. The manipulations required to join the connectors can be performed in conventional fashion via a surgical approach. However, a preferred route is to load the connectors into a sheath or introducer system, and introduce them to the desired location using a conventional percutaneous entry technique, such as the well-known Seldinger technique. For applications in the circulatory system, ultrasound or another guidance system can be utilized to help with the needle, wire guide, and dilator cannulation of the vessel before placement. This could be done, for example, on both the artery and the vein.

Once the connectors are sutured or otherwise attached to the vessels as described, the screw connection is established between the two connectors. With the embodiment of FIG. 1, one or both of the vessels is preferably pre-torqued to avoid the circumferential twist of one or both vessels. As stated, such pre-torque is not generally necessary with the embodiments of FIGS. 2 and 3.

All of the synthetic components described herein are formed of biologically compatible conventional materials having sufficient strength for the purposes described. Preferably, the tubular connections are formed from a rigid or semi-rigid plastic suitable for implantation into a human or other animal.

Although the inventive apparatus has been described for use in joining two blood vessels, those skilled in the art will recognize that other known components can likewise be joined, such as other vessels, other body structures wherein a pathway is to be established (or re-established) therebetween, synthetic graft materials, exogenous materials, ands combinations of them. Likewise, a blood vessel may be attached to a synthetic graft vessel or an exogenous vessel. In addition, the invention is not limited to vascular access, but rather, may also include other applications. Non-limiting examples of such applications include bypass grafting between two blood vessels, including fem-fem (femoral artery and femoral vein) and fem-pop; coronary artery bypass grafting; and shunting outside of the circulatory system to help alter flow of fluid including gastrointestinal tract (e.g., liver and gall bladder), the urinary system (e.g., ureter and urethra), beyond the blood-brain barrier (e.g., for hydroencephalopathy), and in the reproductive system (e.g., ovarian recannulation).

It is therefore intended that the foregoing detailed description be regarded as illustrative rather than limiting, and that it be understood that it is the following claims, including all equivalents, that are intended to define the spirit and scope of this invention.

1. A system for providing an end-to-end anastomosis connection between two vessels, comprising:
a first hollow tube having two ends, one end of said first hollow tube attachable to a free end of a first one of said vessels, the other end comprising a first connector;
a second hollow tube having two ends, one end of said second hollow tube attachable to a free end of a second one of said vessels, the other end comprising a second connector,
said first connector and said second connector being sized and shaped such that a mating connection can be established therebetween.

2. The system of claim 1, wherein said first and second connectors comprise mating screw threads.

3. The system of claim 1, wherein said one end of at least one of said first and second hollow tubes is attachable to said free vessel end by one or more sutures.

4. The system of claim 1, wherein at least one of said vessels comprises a vessel selected from the group consisting of blood vessels, synthetic vessels and exogenous vessels.

5. The system of claim 1, wherein at least one of said hollow tubes includes a rotatable structure for permitting at least partial rotation of said one hollow tube relative to the other of said hollow tubes.

6. The system of claim 5, wherein said rotatable structure comprises a ball bearing ring.

7. The system of claim 1, wherein said first connector comprises a plurality of hook connectors, and said second connector comprises a plurality of loop connectors.

8. The system of claim 1, wherein said first connector comprises external screw threads and said second connector comprises internal screw threads.

9. A system for establishing an end-to-end anastomosis connection between two vessels, comprising:
a first ring member, said first ring member connected to a free end of a first one of said vessels, said first ring member including a plurality of hook connectors;
a second ring member, said second ring member connected to a free end of a second one of said vessels, said second ring member including a plurality of loop connectors;
said hook and loop connectors being sized and oriented such that a linkable connection can be established therebetween, said linkable connection for establishing fluid communication between said first and second vessels.

10. The system of claim 9, wherein at least one of said first and second hollow ring members is attachable to said free vessel end by suturing.

11. A method for joining two vessels in an end-to-end anastomosis connection to establish a fluid pathway therebetween, comprising:
providing a connection system for said vessels, said connection system comprising a first annular member having two ends and a second annular member having two ends, one end of said first annular member attachable to a free end of a first one of said vessels, and one end of said second annular member attachable to a free end of a second one of said vessels, the other end of said first annular member including a first connection member and the other end of said second connection member including a second connection member, said first and second connection members being sized and shaped for establishing a mating connection therebetween;
attaching said one end of said first annular member to said first vessel free end;
attaching said one end of said second annular member to said second vessel free end; and
establishing said mating connection between said first and second connection members.

12. The method of claim 11, wherein at least one of said first and second annular members is attached by suturing said at least one annular member to said vessel free end.

13. The method of claim 11, wherein said first and second annular members comprise respective first and second tubular conduits, one of said tubular conduits comprising external screw threads and the other tubular conduit comprising internal screw threads, and wherein said mating connection comprises mated external and internal screw threads.

14. The method of claim 13, including the steps of pre-torqueing at least one of said tubular conduits prior to establishing said mating connection, and releasing said pre-torque, such that said at least one conduit rotates relative to the other conduit such that said threads form said mating connection, thereby establishing said fluid pathway.

15. The method of claim 13, wherein at least one of said hollow tubes includes a rotatable structure for permitting at least partial rotation of said one hollow tube relative to the other of said hollow tubes for establishing said mating connection.

16. The method of claim 15, wherein said rotatable structure comprises a ball bearing ring.

17. The method of claim 11, wherein said first connection member comprises a plurality of hook connectors, and said second connection member comprises a plurality of loop connectors.
